(19) United States
(12) Patent Application Publication
(10) Pub. No.: US 2009/0247885 A1
(43) Pub. Date: Oct. 1, 2009

(54) PULSE WAVE MEASURING APPARATUS AND AUTONOMIC NERVOUS ANALYSIS SYSTEM HAVING THE SAME

(75) Inventors: Takuji Suzuki, Yokohama-shi (JP); Kazushige Ouchi, Saitama-shi (JP); Kenichi Kameyama, Kawasaki-shi (JP)

Correspondence Address: TUROCY & WATSON, LLP, 127 Public Square, 57th Floor, Key Tower, CLEVELAND, OH, 44114, US

(73) Assignee: KABUSHIKI KAISHA TOSHIBA, Tokyo (JP)

(21) Appl. No.: 12/405,422

(22) Filed: Mar. 17, 2009

(30) Foreign Application Priority Data
Mar. 25, 2008 (JP) 2008-078734

(51) Int. Cl. A61B 5/02

(52) U.S. Cl. 600/500; 600/504

(57) ABSTRACT

A pulse wave measuring apparatus includes a sensor unit, a light emitting unit provided on the sensor unit and configured to emit light to a target region that is a body part of a subject, a light receiving unit provided on the sensor unit and configured to receive the light reflected from the target region to generate a received light signal that represents a change in blood flow in the target region, a measuring unit having a principal surface, holding the sensor unit on the principal surface and configured to measure a pulse wave of the subject, from the change in blood flow in the target region, a belt being expandable and configured to wrap around the target region to hold the measuring unit, with the principle surface facing the target region, and an elastic member arranged on the principle surface to surround the sensor unit.

### CROSS-REFERENCE TO RELATED APPLICATIONS

This application is based upon and claims the benefit of priority from prior Japanese Patent Application No. 2008-078734, filed Mar. 25, 2008, the entire contents of which are incorporated herein by reference.

### BACKGROUND OF THE INVENTION

1. Field of the Invention

The present invention relates to a pulse wave measuring apparatus that is designed to be attached to one body part of a subject and to measure the pulse wave in the subject, and also to an autonomic nervous analysis system that incorporate the pulse wave measuring apparatus.

2. Description of the Related Art

In various medical examinations performed in medical facilities such as hospitals, a pulse wave is measured from changes in the blood flow in a body part of a subject, particularly in a peripheral portion of the subject. Based on the peak of the acceleration pulse wave, i.e., the result of 2nd-order differentiation of the pulse wave, the degree of the subject's arteriosclerosis and the subject's blood-vessel age is determined. The pulse rate meter is used in daily life to display the pulse wave of the subject who is running. The measured pulse wave is also used to monitor the safety of, for example, elderly people who live alone or infants. The measured pulse wave is also used to measure sleep condition of the subject.

An ordinary pulse wave measuring apparatus comprises a pulse wave sensor and a main unit. The main unit may be attached to, for example, the wrist of a subject, and the pulse wave sensor is attached to the subject's finger tip or finger pad. The main unit measures the pulse wave of the subject, from the signal the pulse wave sensor detects. The pulse wave sensor is, for example, a photoplethysmographic sensor. The photoplethysmographic sensor includes a light-emitting element and a light-receiving element. The light-emitting element emits light of a relatively short wavelength (about 440 to 550 nm). The light is reflected by the subject's dermis and caught by the light-receiving element. Thus, the change in blood flow in the capillaries lying in the dermis is detected. The photoplethysmographic sensor is advantageous over the pulse wave sensor based on a pressure sensor, in that the position of the subject's blood vessel need not be determined, no sensors need be positioned or the influence from the shift of the sensor is little.

If the monitoring of an elderly person or an infant must be performed every day over an extended period, however, the pulse wave sensor will restrict the actions the subject performs in daily life, such as washing hands and picking up objects. In view of this, the pulse wave sensor should not remain attached to the subject's finger for a long time in order to continue measurement of the subject's pulse wave over an extended period. Further, the pulse wave sensor attached to the finger is susceptible to external force. The artifacts by an external force may adversely influence the pulse wave measurement.

Attempts have been made to attach the pulse wave sensor to the back of the main unit of the pulse wave measuring apparatus and to measure a pulse wave from the changes in blood flow in the wrist of the subject. This is indeed more appropriate to the everyday use of the pulse rate meter than to attach the pulse wave sensor to the subject's finger. However, the signal generated from the reflected light the light-receiving element has received is not sufficiently intense, inevitably because the blood capillary density is lower in the wrist than in the finger. To make matters worse, the pulse wave sensor is more likely to move on the wrist than on the finger, because the wrist has greater freedom of movement than the finger. This fails to ensure stability of measurement. Furthermore, if the pulse wave measuring apparatus grips the wrist while it is continuously measuring the pulse wave over an extended period (for example, while the subject is sleeping), the blood will stay in the wrist, which may decrease the intensity of the reflected light.

JP No. 3722203 describes a pulse wave sensor, and JP No. 3803351 describes a pulse wave data acquisition apparatus. In the pulse wave sensor and the pulse wave data acquisition apparatus, the sensor head convexly projects from the back of the main unit, preventing the blood from staying in the part of the subject being measured. JP-U H8-10301 discloses a pulse wave detector that has a pressure sensor, which detects changes in pressure in the arteries existing in the target region. The pulse wave detector described in JP-U H8-10301 detects a pulse wave from the changes in pressure. The pressure sensor is surrounded by an elastic member, which stabilizes the pressure applied to the target region.

In the pulse wave sensor disclosed in JP No. 3722203 and pulse wave data acquisition apparatus disclosed in JP No. 3803351, the sensor head convexly projects. Inevitably, the sensor head is less stable than in the case where it lies flat on the back of the main unit. That is, the sensor head is likely to move relative to the target region of the subject when the subject tosses about in bed or when the main unit receives an external force. If the sensor head moves so, the angle of incidence at which the light is emitted from the light-emitting element to the subject's wrist will change. As the angle of incidence so changes, the intensity of the optical signal the light-receiving element receives changes greatly. In view of this, the pulse wave sensor and pulse wave data acquisition apparatus disclosed in JP No. 3722203 and JP No 3803351, respectively, are disadvantageous to the stability of continuous measurement of a pulse wave over an extended period.

In the pulse wave detector disclosed in JP-U H8-010301, the elastic member indeed surrounds the pressure sensor in order to stabilize the pressure with which the sensor pushes against the target region of the subject. However, the sensor head cannot stably remain at a desired position relative to the target region of the subject.

### BRIEF SUMMARY OF THE INVENTION

According to an aspect of the invention, there is provided a pulse wave measuring apparatus comprising: a sensor unit; a light emitting unit provided on the sensor unit and configured to emit light to a target region that is a body part of a subject; a light receiving unit provided on the sensor unit and configured to receive the light reflected from the target region to generate a received light signal that represents a change in blood flow in the target region; a measuring unit having a principal surface, holding the sensor unit on the principal surface and configured to measure a pulse wave of the subject, from the change in blood flow in the target region; a belt being expandable and configured to wrap around the target region to hold the measuring unit, with the principle surface facing the target region; and an elastic member arranged on the principle surface to surround the sensor unit.

According to another aspect of the invention, there is provided a pulse wave measuring apparatus comprising: a sensor unit; a light emitting unit provided on the sensor unit and configured to emit light to a target region that is a body part of a subject; a light receiving unit provided on the sensor unit and configured to receive the light reflected from the target region to generate a received light signal that represents a change in blood flow in the target region; an elastic member having a concavity and supporting the sensor unit in the concavity; a measuring unit having a principal surface, holding the elastic member on the principal surface and configured to measure a pulse wave of the subject, from the change in blood flow in the target region; and a belt being expandable and configured to wrap around the target region to hold the measuring unit, with the principle surface facing the target region.

According to another aspect of the invention, there is provided a pulse wave measuring apparatus comprising: a sensor unit; a light emitting unit provided on the sensor unit and configured to emit light to a target region that is a body part of a subject; a light receiving unit provided on the sensor unit and configured to receive the light reflected from the target region to generate a received light signal that represents a change in blood flow in the target region; a measuring unit having a principal surface and configured to measure a pulse wave of the subject, from the change in blood flow in the target region; an elastic member arranged on the principal surface and having an end portion that faces the target region; a plate-like member arranged on the end portion and combined with the sensor unit; and a belt being expandable and configured to wrap around the target region to hold the measuring unit, with the principle surface facing the target region.

According to another aspect of the invention, there is provided pulse wave measuring apparatus comprising: a sensor unit; a light emitting unit provided on the sensor unit and configured to emit light to a target region that is a body part of a subject; a light receiving unit provided on the sensor unit and configured to receive the light reflected from the target region to generate a received light signal that represents a change in blood flow in the target region; a supporting member having a principal surface and supporting the sensor unit on the principal surface; a belt being expandable and configured to wrap around the target region to hold the supporting member, with the principle surface facing the target region; an elastic member arranged on the principal surface and surrounding the sensor unit; and a measuring unit configured to measure a pulse wave of the subject, from the change in blood flow in the target region.

### BRIEF DESCRIPTION OF THE SEVERAL VIEWS OF THE DRAWING

FIG. 1 is a plane view showing a pulse wave measuring apparatus according to an embodiment;

FIG. 2 is a sectional view taken along line II-II′ of FIG. 1;

FIG. 3 is a rear view of the pulse wave measuring apparatus of FIG. 1;

FIG. 4 is a block diagram showing the configuration of the main unit of the pulse wave measuring apparatus shown in FIG. 1;

FIG. 5 is a block diagram showing an autonomic nervous analysis system that incorporates the pulse wave measuring apparatus according to the embodiment;

FIG. 6A is a diagram showing the configuration of a comparative pulse wave measuring apparatus;

FIG. 6B is a diagram showing the configuration of a comparative pulse wave measuring apparatus;

FIG. 6C is a diagram showing the configuration of a comparative pulse wave measuring apparatus;

FIG. 7 is a diagram explaining the advantage the configuration shown in FIG. 2 achieves;

FIG. 8 is a diagram showing a pulse wave measuring apparatus that differs from the apparatus of FIG. 2;

FIG. 9 is a diagram showing a pulse wave measuring apparatus that differs from the apparatuses of FIGS. 2 and 8; and

FIG. 10 is a diagram showing a pulse wave measuring apparatus that differs from the apparatus of FIGS. 2, 8 and 9.

### DETAILED DESCRIPTION OF THE INVENTION

An embodiment of the present invention will be described with reference to the accompanying drawings.

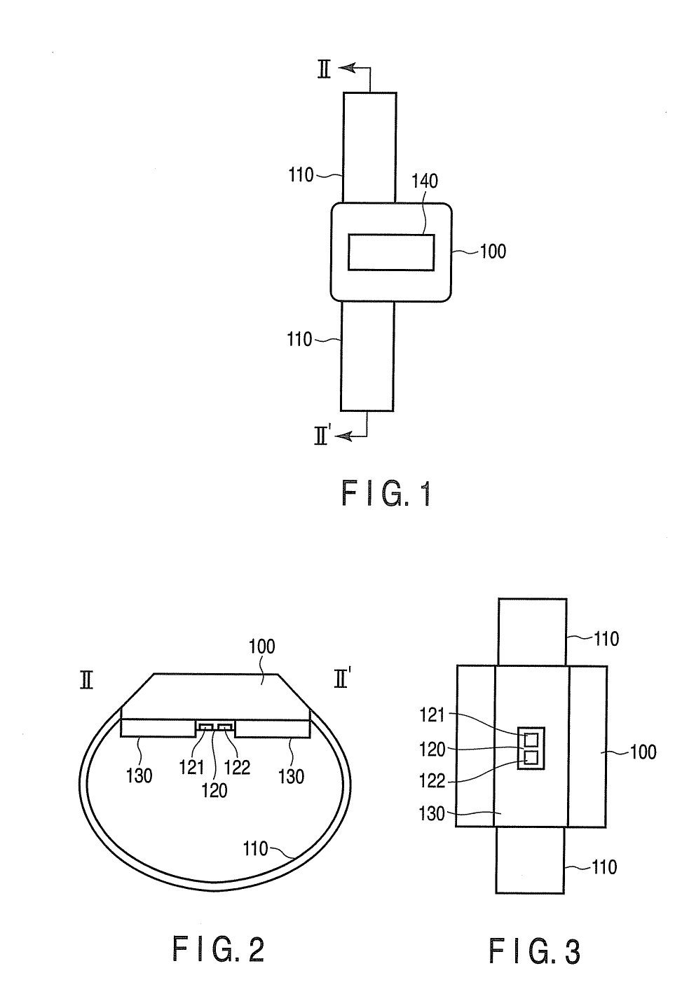

As FIG. 1 shows, a pulse wave measuring apparatus according to an embodiment of this invention is shaped like a wristwatch. The apparatus comprises a main unit (measuring unit) 100 and a belt 110. The measuring unit 100 has a display unit 140. The belt 110 is fastened to the measuring unit 100. The belt 110 may be wrapped around the target region of a subject, whereby the apparatus is attached to the subject. The target region of the subject is, for example, the wrist.

The belt 110 wrapped around the target region holds the measuring unit 100, the back facing the wrist. The belt 110 is made of high-elasticity resin material such as urethane foam rubber, neoprene rubber used for diving suits, or viscoeleastic foam used for small-rebound pillows. The belt 110 may have hook-and-loop fasteners on both end portions. The hook-and-loop fasteners may be wrapped around the subject's wrist to overlap each other, whereby the pulse wave measuring apparatus is appropriately attached to the target region of the subject.

As FIG. 2 shows, a sensor unit 120 convexly projects from the back of the measuring unit 100. The sensor unit 120 is an photoplethysmographic sensor and includes a light-emitting unit 121 and a light-receiving unit 122. The light-emitting unit 121 is a green LED (wavelength of about 520 nm) or a blue LED. More specifically, the light receiving unit 122 is a photodiode device. The light emitting unit 121 emits light to the target region of the subject. The light receiving unit 122 receives the light reflected from the target region and detects the changes in intensity of the light resulting from the changes in blood flow. The unit 122 then converts these changes in intensity of light into an electric current.

An elastic member 130 is provided around the sensor unit 120. It is desired that the elastic member 130 be made high-elasticity resin material such as urethane foam rubber, neoprene rubber, or viscoeleastic foam. The elastic member 130 prevents the sensor unit 120 from changing in position relative to the target region of the subject. That is, the elastic member 130 absorbs the external force applied to the measuring unit 100 and the motion of the subject. It is therefore desired that the circumferential dimension of the elastic member 130 should be almost the same as the circumferential dimension of the measuring unit 100. Further, the circumferential dimension of the elastic member 130 is preferably greater than the axial dimension, because the positional change of the elastic member 130 tends to be greater circumferentially than axially.

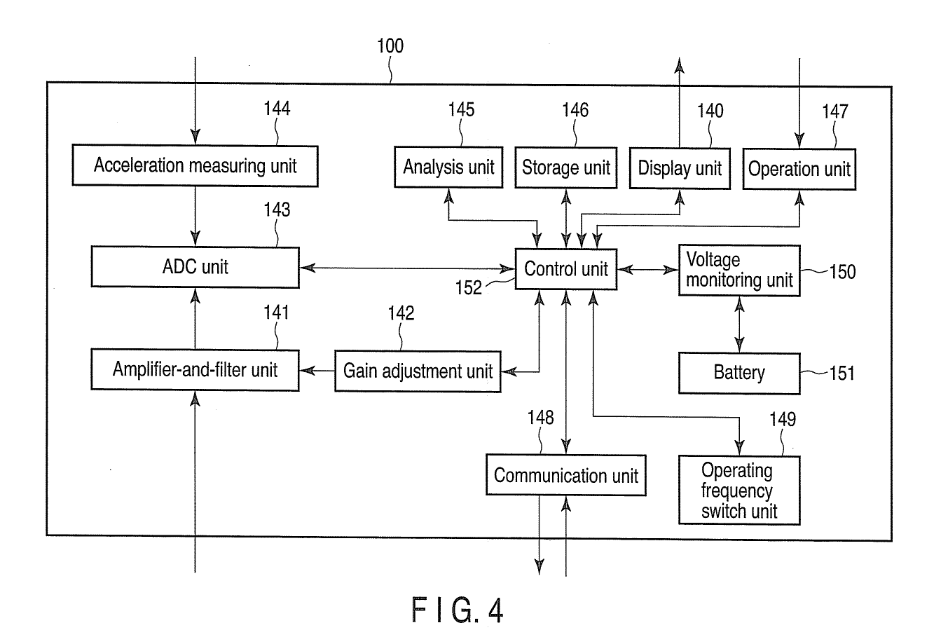

As FIG. 4 shows, the measuring unit 100 of the pulse wave measuring apparatus according to this embodiment has a display unit 140, an amplifier-and-filter unit 141, a gain adjustment unit 142, an analog-to-digital conversion unit (ADC unit) 143, an acceleration measuring unit 144, an analysis unit 145, a storage unit 146, an operation unit 147, a communication unit 148, an operating-frequency switch unit 149, a voltage monitoring unit 150, a battery 151, and a control unit 152.

The display unit 140 is provided on the front of the measuring unit 100, thus facing away from the back of the measuring unit 100. The display unit 140 displays various data items to the subject. The unit 140 is a liquid display (LCD) of the type used in wrist watches, or an organic electroluminescent display. The data items the unit 140 can display are, for example, the present time, the beats per minute of the subject, pulse wave data, the amount of power remaining in the battery 151, the state of the storage unit 146, the state of communication, and various data items acquired through the analysis of the pulse wave data acquired from the subject. The display unit 140 may display these data items at a time, or one by one in accordance with the instructions the subject inputs at the operation unit 147 as will be described later or with the instructions received from the control unit 152.

The light receiving unit 122 incorporated in the sensor unit 120 supplies a current to the amplifier-and-filter unit 141. As indicated above, the current supplied from the light receiving unit 122 to the amplifier-and-filter unit 141 represents the change in blood flow in the target region of the subject. The amplifier-and-filter unit 141 comprises a current-to-voltage converter, a variable gain amplifier (VGA), and a filter. The current-to-voltage converter converts the current to a voltage signal. The variable gain amplifier amplifies the voltage signal. The filter limits the band of the voltage signal. More precisely, a high-pass filter (having a cutoff frequency of, for example, 0.1 Hz) and a low-pass filter (having a cutoff frequency of, for example, 50 Hz) limits the band of the amplified voltage signal. The filter may be replaced by a band-pass filter. The voltage signal thus limited in band is input to the ADC unit 143.

The ADC unit 143 is an analog-to-digital converter (ADC) that has, for example, 10-bit resolution. The ADC conversion unit 143 performs analog-to-digital conversion on the voltage signal input from the amplifier-and-filter unit 141, generating pulse wave data. The pulse wave data is input from the ADC conversion unit 143 to the control unit 152. Further, the ADC conversion unit 143 performs analog-to-digital conversion on a signal input from the acceleration measuring unit 144 and representing three-axis acceleration, generating acceleration data. The acceleration data is input to the control unit 152, too.

In accordance with the instructions coming from the control unit 152, the gain adjustment unit 142 adjusts the gain of the VGA provided in the amplifier-and-filter unit 141. More specifically, the gain adjustment unit 142 increases the gain if the amplitude of the pulse data received from the ADC unit 143 is smaller than a first threshold value. If the amplitude of the pulse data is greater than a second threshold value that is greater than the first threshold value, the gain adjustment unit 142 decreases the gain.

The acceleration measuring unit 144 is, for example, an accelerometer that measures the accelerations (e.g., −2 G to +2 G) with which the measuring unit 100 moves along the three axes (x-, y- and z-axes). The accelerometer 104 measures the accelerations of the measuring unit 100 at intervals of, for example, 50 ms, generating analog data representing the accelerations at which the subject's wrist moves. The analog data is input to the ADC conversion unit 143. The accelerations the acceleration measuring unit 144 has measured include not only the dynamic acceleration of the wrist, but also the static acceleration (i.e., the acceleration due to gravity). Note that the analog data representing the accelerations measured may be adjusted in terms of offset or gain before it is input to the ADC conversion unit 143.

The analysis unit 145 acquires the acceleration data representing the accelerations along thee axes or the pulse wave data through the control unit 152 and then performs various analyses based on the data. For example, the analysis unit 145 may analyze the motion of the subject's wrist from the acceleration data. Further, the analysis unit 145 may determine the sleep/arousal timing of the subject from the motion amount, thereby finding the subject's sleep hours. Moreover, the analysis unit 145 may acquire the pulse wave data through the control unit 152 and determine the pulse interval from the pulse wave data.

The storage unit 146 may have a flash memory for storing the results of various analyses the analysis unit 145 has performed (e.g., the history of sleep hours, the pulse interval data, the motion of the subject's wrist, etc.), which have been supplied through the control unit 152. The storage unit 146 may store also the pulse wave data and acceleration data, both coming from the ADC conversion unit 143 through the control unit 152.

When operated by the subject, the operation unit 147 generates instructions. The instructions are supplied to the control unit 152. The operation unit 147 has push-button switches including a mode switch and a backlight switch. The mode switch may be pushed to switch the operating mode of the measuring unit 100. The backlight switch may be pushed to turn on the backlight of the display unit 140.

The communication unit 148 is configured to transmit various data items stored in the storage unit 146 from the measuring unit 100 via a wired network or a radio network and to receive data transmitted from outside the measuring unit 100. To be more specific, the communication unit 148 uses the Universal Serial Bus (USB) or short-range wireless communication network, accomplishing communication with personal computers (PCs), personal digital assistants (PDAs), cellular telephones, or the like.

The operating-frequency switch unit 149 changes over the frequency of the control clock to supply to some other components of the measuring unit 100, in accordance with the operating mode of the measuring unit 100. That is, the measuring unit 100 may operate not only in the pulse wave measuring mode, but also in other various operating modes such as the time measuring mode of ordinary wrist watches. More precisely, the operating-frequency switch unit 149 lowers the operating frequency when the operating mode of the measuring unit 100 is switched from the pulse wave measuring mode to the time measuring mode, thereby reducing power consumption. When the operating mode of the measuring unit 100 is switched from the time measuring mode to the pulse wave measuring mode, the operating-frequency switch unit 149 increases the operating frequency, thereby ensuring a sufficiently high signal-processing speed.

The battery 151 supplies power to the sensor unit 120 and the components of the measuring unit 100. The voltage monitoring unit 150 keeps monitoring the discharge voltage of the battery 151, notifying the control unit 152 of the data that represents the amount of power remaining in the battery 151. This data may be displayed by the display unit 140 at all times or only when the amount of power falls below a prescribed value, or may not be displayed at all.

The control unit 152 controls some other components of the measuring unit 100. More specifically, the control unit 152 exchanges data with the other components of the measuring unit 100 and gives instructions to the other components, causing them to perform various processes.

An autonomic nervous analysis system that incorporates the pulse wave measuring apparatus according to this embodiment will be described. This system is designed to acquire an autonomic nervous index showing the state of the subject's autonomic nervous system, from the pulse wave data acquired from the subject while the subject is sleeping.

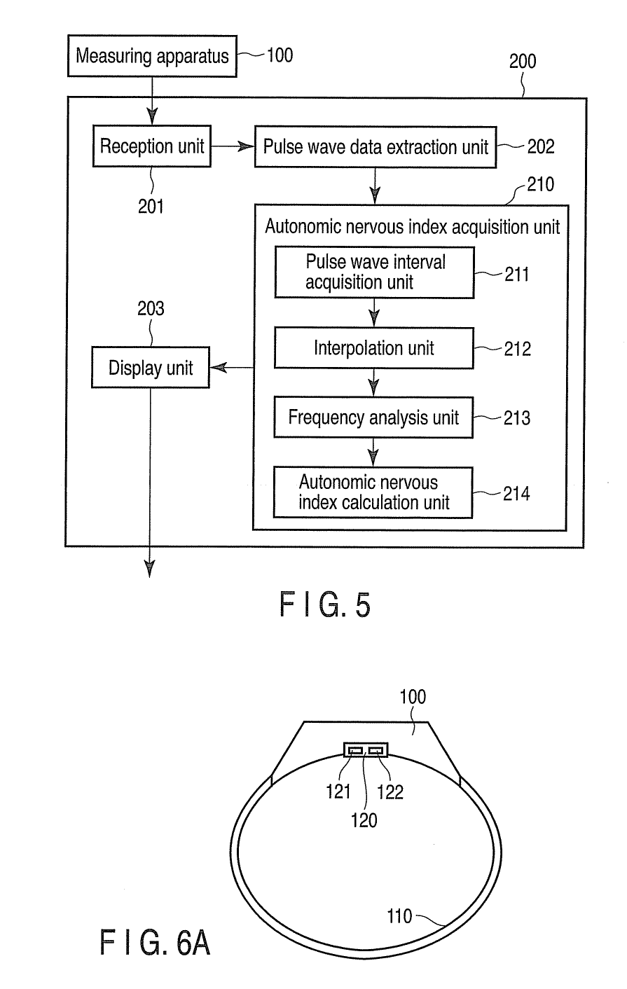

The autonomic nervous analysis system includes an autonomic nervous analysis apparatus 200. As shown in FIG. 5, the autonomic nervous analysis apparatus 200 has a reception unit 201, a pulse wave data extraction unit 202, a display unit 203, and an autonomic nervous index acquisition unit 210. The apparatus 200 performs a sequence of processes, as described below, as a PC, for example, executes a specific analysis program.

The reception unit 201 receives data transmitted from the communication unit 148 of the measuring unit 100 and containing the pulse wave data. This data thus received is input, as received data, to the pulse wave data extraction unit 202. Note that the communication unit 148 may transmit the pulse wave data items, one by one, or in units each consisting of a particular number of wave data items.

The pulse wave data extraction unit 202 extracts the pulse wave data from the data the reception unit 202 has received. The pulse wave data thus extracted is input to the autonomic nervous index acquisition unit 210. The display unit 203 is, for example, an LCD or an organic electroluminescent display and displays the autonomic nervous index supplied from the autonomic nervous index acquisition unit 210, which will be described later.

The autonomic nervous index acquisition unit 210 acquires, as autonomic nervous index, the sympathetic nervous index LF indicating the activity of the sympathetic nerve and the parasympathetic nervous index HF indicating the activity of the parasympathetic nerve. Since the pulse wave is synchronous with the heartbeat, an index representing the state of the autonomic nerve that controls the heartbeat can be obtained from, for example, the pulse intervals detected from the sleeping subject. The autonomic nervous index thus acquired is input to the display unit 203. The autonomic nervous index acquisition unit 210 incorporates a pulse wave interval acquisition unit 211, an interpolation unit 212, a frequency analysis unit 213, and an autonomic nervous index calculation unit 214, which will be described in the order they are mentioned.

The pulse wave interval acquisition unit 211 processes the pulse wave data about the subject, input from the reception unit 202, and acquires pulse interval data, as will be explained below.

First, the pulse wave interval acquisition unit 211 samples the pulse wave data, thus performing time differentiation, and removes the DC fluctuation components from the pulse wave data. Next, the pulse interval acquisition unit 211 sets, as threshold value for detecting the pulse interval, a specific value between the maximum and minimum values in a period starting about one second before a pulse wave sampling point and ending about one second after the pulse wave sampling point. The threshold value is, for example, the sum of the minimum value and 90% of the difference (amplitude) between the maximum value and the minimum value. The pulse wave interval acquisition unit 211 then determines the points when the sampled pulse wave data surpasses the threshold value after the DC fluctuation components have been removed from it. The period between any two adjacent points thus determined is input, as pulse wave interval (RR interval) data, to the interpolation unit 212.

The interpolation unit 212 interpolates the pulse interval data input from the pulse wave interval acquisition unit 211, and then re-samples the same. The pulse interval data acquired by the pulse wave interval acquisition unit 211 represents irregular intervals that are equivalent to the intervals of the pulse waves detected from the subject. This is why the interpolation unit 212 re-samples the pulse interval data, changing the same to data representing regular intervals, so that the frequency analysis unit 213 may perform an effective frequency analysis. To be more specific, the interpolation unit 212 generates, for example, one-minute data sets from the pulse interval data. The interpolation unit 212 then interpolates the pulse interval data set with a high-order polynomial. For example, the interpolation unit 212 performs a third-order polynomial interpolation on the pulse interval data, using each interpolation target point and two points preceding and following the interpolation target point, respectively, and then re-samples the pulse interval data, thereby providing pulse interval data that represents regular intervals. The pulse interval data, thus generated through interpolation, is input to the frequency analysis unit 213.

The frequency analysis unit 213 performs fast Fourier transformation (FFT) on the interpolated pulse interval data, in units of data sets, thus converting the data to a frequency spectrum distribution. The frequency spectrum distribution is input to the autonomic nervous index calculation unit 214. The frequency analysis the frequency analysis unit 213 performs is not limited to FFT. Any other method, such as AR mode method, maximum entropy method or wavelet method, may be employed instead. Nonetheless, FFT is desirable because it involves but a relatively small amount of processing data.

The autonomic nervous index calculation unit 214 calculates a power spectrum distribution from the frequency spectrum distribution supplied from the frequency analysis unit 213. The autonomic nervous index calculation unit 214 then calculates a sympathetic nervous index LF from the peak in the low-frequency region (0.05 to 0.15 Hz) of the power spectrum distribution, and a parasympathetic nervous index HF from the peak in the high-frequency region (0.15 to 0.4 Hz) of the power spectrum distribution. More specifically, the unit 214 finds, as sympathetic nervous index LF, the arithmetic mean of the power spectra at the data point representing the peak in the low-frequency region and the two points preceding and following the data point, and finds, as parasympathetic nervous index HF, the arithmetic average of the power spectra at the data point representing the peak in the high-frequency region and the two points preceding and following the data point.

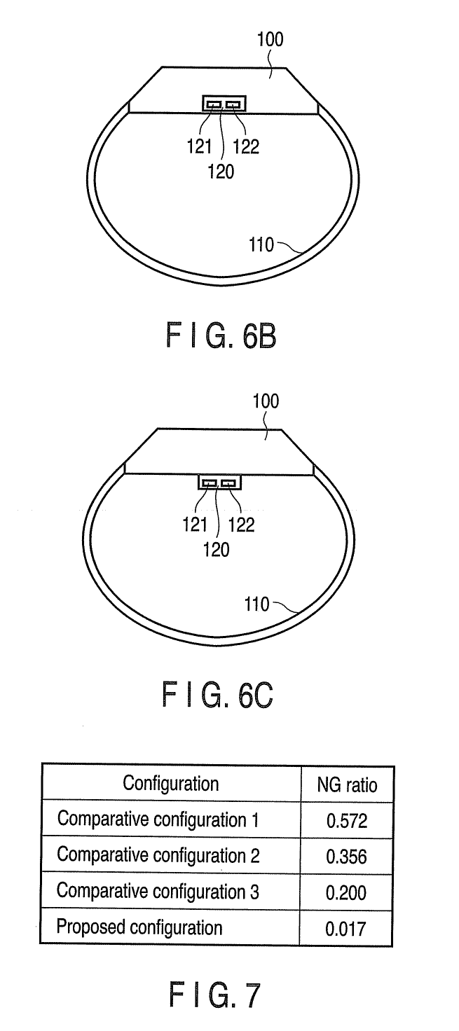

The advantages attained by the configuration shown in FIG. 2 will be explained with reference to FIG. 6A to FIG. 6C.

As described above, the pulse wave measuring apparatus according to this embodiment is configured as shown in FIG. 2. Hereinafter, this specific configuration will be referred to as “proposed configuration.” FIG. 6A shows a comparative configuration 1, in which the measuring unit 100 of the apparatus is R-shaped so that its back may wrap around the subject's wrist and the sensor unit 120 does not project. FIG. 6B shows a comparative configuration 2, in which the measuring unit 100 has a flat back and the sensor unit 120 does not project. FIG. 6C shows a comparative configuration 3, in which the measuring unit 100 has a flat back and the sensor unit 120 projects. At a glance, the comparative configuration 3 may look similar to the proposed configuration, but differs in that no elastic members 130 are arranged around the sensor unit 120.

An experiment was conducted, in which four pulse wave measuring apparatuses that have the proposed configuration and comparative configurations 1, 2 and 3, respectively were used over an extended period in order to evaluate them in terms of accuracy of measurement. Note that the elastic member 130 used in the proposed configuration was made of foamed urethane rubber.

Four subjects were subjected to pulse wave measurement using the four pulse wave measuring apparatuses each for two nights while sleeping (each time, from going to bed in the evening until getting up in the morning). The pulse intervals recorded by each apparatus were commonly analyzed, acquiring the beats per minute (bpm) for each subject. If the beats per minute acquired fell within a range of 40 to 110 bpm for any minute, the one-minute period was evaluated as “correct period (being valid).” If the beats per minute fell outside this range or if the subject moved more than a prescribed amount, for any minute, the one-minute period was evaluated as “NG period (being invalid).” If NG periods continuously lasted for 30 minutes or more, the NG periods were extracted. The ratio of the sum of extracted NG periods to the sleep hours, i.e., the “NG ratio,” was used as an index representing the accuracy of extended pulse wave measurement.

Thus, eight NG ratios were obtained for each pulse wave measuring apparatus (that is, four subjects*two nights). The average value of the eight NG ratios was as shown in FIG. 7. As seen from FIG. 7, the NG ratio of the pulse wave measuring apparatus of the proposed configuration was as less than one-tenth ( 1/10) of the NG ratio of the apparatus of any other configuration (i.e., configuration 1, 2 or 3). This is probably because the apparatus of the proposed configuration can achieve high accuracy of measurement since the elastic member 130 is arranged around the sensor unit 120, keeping the sensor unit 120 parallel to the subject's wrist. That is, the elastic member 130 absorbs the external force applied to the measuring unit 100 and the motion of the subject, reducing the positional shift of the sensor unit 120, relative to the target region of the subject.

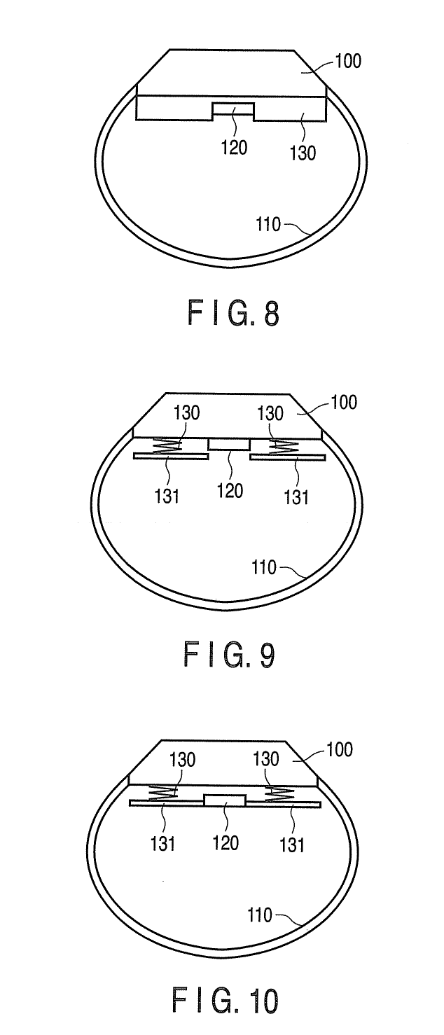

Modifications of the proposed configuration will be described with reference to FIG. 8 to FIG. 10.

In the modification of FIG. 8, an elastic member 130 is further provided between the sensor unit 120 and the back of the measuring unit 100. In other words, the elastic member 130 has a concavity in the surface that faces the target region of the subject. The sensor unit 120 is fitted in the concavity. In the configuration of FIG. 8, too, the elastic member 130 absorbs the external force applied to the measuring unit 100 and the motion of the subject, thereby reducing the positional shift of the sensor unit 120, relative to the target region of the subject.

In the modification of FIG. 9, plate-like members 131 are attached to that surface of the elastic member 130, which face the target region of the subject. The elastic member 130 may be springs, not a member made of high-elasticity resin material. In the modification of FIG. 9, too, the elastic member 130 absorbs the external force applied to the measuring unit 100 and the motion of the subject, reducing the positional shift of the sensor unit 120, relative to the target region of the subject.

In the modification of FIG. 10, plate-like members 131 are attached to that surface of the elastic member 130, which face the target region of the subject, as in the modification shown in FIG. 9. Nonetheless, this modification differs in that the plate-like members 131 is combined with the sensor unit 120. To be more specific, the sensor unit 120 lies flat to the target region of the subject. The elastic member 130 may be springs, not a member made of high-elasticity resin material, as in the modification shown in FIG. 9. In the modification of FIG. 10, too, the elastic member 130 absorbs the external force applied to the measuring unit 100 and the motion of the subject, reducing the positional change of the sensor unit 120, relative to the target region of the subject.

The proposed configuration and the modifications shown in FIGS. 8, 9 and 10 are designed on the assumption that the sensor unit 120 and measuring unit 100 are set in contact with the target region of the subject. In theory, however, the apparatus can measure pulse waves only if the sensor unit 120 is held, facing the target region of the subject. Hence, the measuring unit 100 need not be attached to the subject's wrist. That is, a supporting member that can support the sensor unit 120 may replace the measuring unit 100, and the measuring unit 100 may be separated from the sensor unit 120. Note that the measuring unit 100 and sensor unit 120 are connected by a signal line. The supporting member may be lighter than the measuring unit 100. In this case, the load on the subject's wrist will decrease. The apparatus can therefore be wrapped around the subject's wrist even if the subject is an infant. If the measuring unit 100 is replaced by the supporting member, the positional change of the sensor unit 120, relative to the target region of the subject, will be reduced as in the proposed configuration described above.

As has been described, in the pulse wave measuring apparatus according to the embodiment, the elastic members 130 absorbs the external force applied to the measuring unit 100 and the motion of the subject. Thus, the elastic members 130 reduce the positional change of the sensor unit 120, relative to the target region of the subject. The pulse wave measuring apparatus according to the embodiment can therefore continuously measure the pulse wave of the subject over an extended period reliably.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A pulse wave measuring apparatus comprising:
a sensor unit;
a light emitting unit provided on the sensor unit and configured to emit light to a target region that is a body part of a subject;
a light receiving unit provided on the sensor unit and configured to receive the light reflected from the target region to generate a received light signal that represents a change in blood flow in the target region;
a measuring unit having a principal surface, holding the sensor unit on the principal surface and configured to measure a pulse wave of the subject, from the change in blood flow in the target region;
a belt being expandable and configured to wrap around the target region to hold the measuring unit, with the principle surface facing the target region; and
an elastic member arranged on the principle surface to surround the sensor unit.

2. The apparatus according to claim 1, wherein the elastic member has a circumferential dimension substantially equal to that of the measuring unit.

3. The apparatus according to claim 1, wherein the elastic member has a circumferential dimension greater than an axial dimension.

4. The apparatus according to claim 1, further comprising a plate-like member arranged at that end portion of the elastic member, which faces the target region.

5. A pulse wave measuring apparatus comprising:
a sensor unit;
a light emitting unit provided on the sensor unit and configured to emit light to a target region that is a body part of a subject;
a light receiving unit provided on the sensor unit and configured to receive the light reflected from the target region to generate a received light signal that represents a change in blood flow in the target region;
an elastic member having a concavity and supporting the sensor unit in the concavity;
a measuring unit having a principal surface, holding the elastic member on the principal surface and configured to measure a pulse wave of the subject, from the change in blood flow in the target region; and
a belt being expandable and configured to wrap around the target region to hold the measuring unit, with the principle surface facing the target region.

6. The apparatus according to claim 5, wherein the elastic member has a circumferential dimension substantially equal to that of the measuring unit.

7. The apparatus according to claim 5, wherein the elastic member has a circumferential dimension greater than an axial dimension.

8. The apparatus according to claim 5, further comprising a plate-like member arranged at that end portion of the elastic member, which faces the target region.

9. A pulse wave measuring apparatus comprising:
a sensor unit;
a light emitting unit provided on the sensor unit and configured to emit light to a target region that is a body part of a subject;
a light receiving unit provided on the sensor unit and configured to receive the light reflected from the target region to generate a received light signal that represents a change in blood flow in the target region;
a measuring unit having a principal surface and configured to measure a pulse wave of the subject, from the change in blood flow in the target region;
an elastic member arranged on the principal surface and having an end portion that faces the target region;
a plate-like member arranged on the end portion and combined with the sensor unit; and
a belt being expandable and configured to wrap around the target region to hold the measuring unit, with the principle surface facing the target region.

10. The apparatus according to claim 9, wherein the elastic member is a spring member.

11. A pulse wave measuring apparatus comprising:
a sensor unit;
a light emitting unit provided on the sensor unit and configured to emit light to a target region that is a body part of a subject;
a light receiving unit provided on the sensor unit and configured to receive the light reflected from the target region to generate a received light signal that represents a change in blood flow in the target region;
a supporting member having a principal surface and supporting the sensor unit on the principal surface;
a belt being expandable and configured to wrap around the target region to hold the supporting member, with the principle surface facing the target region;
an elastic member arranged on the principal surface and surrounding the sensor unit; and
a measuring unit configured to measure a pulse wave of the subject, from the change in blood flow in the target region.

12. The apparatus according to claim 11, wherein the elastic member has a circumferential dimension substantially equal to that of the measuring unit.

13. The apparatus according to claim 11, wherein the elastic member has a circumferential dimension greater than an axial dimension.

14. The apparatus according to claim 11, further comprising a plate-like member arranged at that end portion of the elastic member, which faces the target region.

15. An autonomic nervous analysis system comprising:
the apparatus according to claim 1;
an acquisition unit configured to acquire pulse interval data about the subject from the pulse wave measured by the apparatus;
a frequency analysis unit configured to perform frequency analysis on the wave interval data to obtain a frequency spectrum distribution; and
a calculation unit configured to calculate a first index representing an activity of a sympathetic nerve of the subject from a peak in a low-frequency region of a power spectrum distribution corresponding to the frequency spectrum distribution, and a second index representing an activity of a parasympathetic nerve of the subject from a peak in a high-frequency region of the power spectrum distribution.
